(19) United States
(12) Patent Application Publication
(10) Pub. No.: US 2014/0275797 A1
(43) Pub. Date: Sep. 18, 2014

(54) Expandable Cannula with Distal Locking Mechanism

(71) Applicant: IB Medical, LLC, Longmont, CO (US)

(72) Inventors: Zaki G. Ibrahim, Greenwood Village, CO (US); Theodore P. Bertele, Longmont, CO (US)

(73) Assignee: IB Medical, LLC, Longmont, CO (US)

(21) Appl. No.: 14/214,020

(22) Filed: Mar. 14, 2014

Related U.S. Application Data
(60) Provisional application No. 61/799,631, filed on Mar. 15, 2013.

(51) Int. Cl. A61B 17/34

(52) U.S. Cl. CPC A61B 17/3439
USPC 600/208

(57) ABSTRACT

An expandable cannula includes a proximal end portion having a substantially constant diameter therethrough, and a distal end portion, coupled to the proximal end portion and having a proximal end and a distal end. The distal end portion includes a plurality of panels hinged by “living hinges” to the proximal end portion, and a locking mechanism that stabilizes the distal end portion in one of a contracted position and an expanded position. The locking mechanism may be a strut pressed overcenter during expansion distal portion to expanded configuration, or may include a locking ring and a catch for stabilizing the ring in a configuration where it holds the panels in expanded configuration.

### RELATED APPLICATIONS AND PRIORITY CLAIM

The present application claims priority to U.S. Provisional Patent Application 61/799,631 filed 15 Mar. 2013, incorporated herein by reference.

### BACKGROUND

Traditional “open” surgical approaches to the spine and other organs typically involve extended longitudinal incisions, significant tissue disruption, and substantial blood loss. Recovery from these procedures may be prolonged and may involve significant morbidity as patients cope with rehabilitating damaged and atrophied muscle and scar tissue. In addition to the above intraoperative difficulties and problems with rehabilitation, there is increasing evidence that “open” approaches may significantly devitalize tissue, predisposing to significant rates of infection.

In response to these problems and as a direct result of rising healthcare costs, increasing pressure to reduce hospital stays and improve patient recovery, physicians have expressed significant interest in performing surgical procedures through less invasive techniques. Minimally invasive surgery (MIS) is a term which encompasses a wide range of surgical interventions. These surgical interventions involve accomplishing a surgical goal similar to that of a traditional “open” technique using a technique which involves less disruptive surgical dissection. Examples of MIS include laparoscopic and arthroscopic surgical interventions which typically use several small incisions as opposed to a single larger incision.

In no medical subspecialty has this type of approach sparked more interest than in that of spinal surgery. MIS approaches have been at the forefront of much recent literature. MIS appears to substantially decrease blood loss, complications, recovery times and hospital stays in comparison to traditional methods for procedures such as discectomy, decompression, and cervical and lumbar fusions.

### SUMMARY OF THE INVENTION

In an embodiment, an expandable cannula includes a proximal end portion having a substantially constant diameter therethrough, and a distal end portion, coupled to the proximal end portion and having a proximal end and a distal end. The distal end portion includes a plurality of panels hinged by “living hinges” to the proximal end portion, and a locking mechanism that stabilizes the distal end portion in one of a contracted position and an expanded position.

In an embodiment, a method gaining access to a surgical site includes: creating a surgical wound; dissecting and dilating the wound to permit insertion of an expandable distal end portion of a cannula; the cannula having a proximal end portion having a substantially constant diameter therethrough; the distal end portion including a plurality of panels, where the panels are coupled to the proximal end portion by hinges formed of thin flexible plastic simultaneously with molding the panels; expanding the distal end portion into an expanded configuration to displace tissue and provide access through the cannula to the surgical site; and locking the distal end portion in the expanded configuration with a mechanical locking mechanism that stabilizes the distal end portion in the expanded configuration

### BRIEF DESCRIPTION OF THE FIGURES

FIG. 1 depicts an exemplary perspective view of an expandable cannula with distal locking mechanism.

FIG. 2 depicts a perspective view of an exemplary distal locking portion including a plurality of panels in a partially expanded position, in one embodiment.

FIG. 3 depicts the distal locking portion of FIG. 2 in a collapsed position.

FIG. 4 depicts a fold out view of the distal locking portion panels of FIG. 2.

FIG. 5 is a perspective view of an embodiment implemented as a forked, molded, plastic cannula with two integral leaves, configured such that the leaves may have rivets to serve as a locking mechanism, in as-molded configuration.

FIG. 6 illustrates the embodiment of FIG. 5, in expanded configuration.

FIG. 6A is a cross section taken through rivet 620 of the embodiment of FIG. 5 or 6.

FIG. 7 is an exterior view of an embodiment having a locking ring positionable on a ladder lock element, in collapsed position.

FIG. 8 illustrates a cut-away view of the embodiment of FIG. 7, in collapsed position.

FIG. 9 depicts a perspective cut away view of an exemplary locking mechanism including a locking ring that is positionable on a ladder lock element, in expanded position.

FIG. 10 depicts a side cut away view of the locking mechanism of FIG. 9.

FIG. 11 depicts a perspective cut away view of an exemplary locking mechanism including a locking ring that is positionable on a ladder lock of zip-tie style, in one embodiment.

FIG. 12 depicts an exemplary distal end portion 1210 including two skirt portions respectively, in an alternate embodiment.

FIG. 13 is a perspective view of an alternative distal end having two skirt portions and an internal hinged locking strut.

FIG. 14 is a rear cross sectional view of the embodiment of FIG. 13, illustrating the locking strut with hinges.

FIG. 15 is a bottom view of the embodiment of FIG. 13, illustrating an opening for surgical access between locking struts.

FIG. 16 is a cross sectional diagram of an alternative embodiment of a cannula having an internal hinged locking strut and a forked proximal portion having a slot for retaining the locking strut in an over-centered locked position.

FIG. 17 is a cross sectional diagram of the embodiment of FIG. 16 in expanded configuration having the locking strut in an over-centered locked position.

### DETAILED DESCRIPTION OF THE EMBODIMENTS

Most MIS procedures in spinal surgery involve nerve decompression performed through a cannula or “tube”. Typically a guidewire is placed under x-ray control to localize the problematic level. A small incision is made over the location of the guidewire and tissue dilators are placed prior to the placement of the cannula in order to access the surgical site and dilate the muscular plane, providing minimal tissue disruption while exposing underlying tissue. While the cannula or “tube” is effective in simple procedures such as lumbar laminectomies and discectomies, more extensive access to the surgical site is required for more involved procedures such as interbody fusion or pedicle screw placement.

Accordingly, MIS retractors which enable more extensive exposure have been developed. The majority of these retractors are fixed “bladed” systems. This type of system typically allows for the retractor to be placed into the surgical bed in a “contracted” position. Once placed, the retractor may then open, providing tissue distraction from the surgical site. Several retractors have been developed which also allow the blades of the retractor to “tilt” or allow further retraction on the distal end than the proximal end. These multi-bladed retractors are frequently cumbersome and clumsy to assemble and deploy, adding several steps and valuable time to the surgical procedure. Other disadvantages of these fixed bladed systems include the issue of tissue “creep” between blades of the system when the retractor is in an expanded position. Also, this type of retractor is fixed in position and allows only a direct longitudinal line of sight between the surgeon and the surgical site. Thus, the surgeons view is limited by the amount of tissue retraction proximally.

In the case of minimally invasive spinal surgery (MIS spine surgery), the extent of exposure is typically limited by the fascial layer lying between the subcutaneous and muscular layers (in lumbar surgery this is the lumbodorsal fascia). Thus, fixed, bladed spinal retractors are limited by this fascial layer in providing the surgeon with direct line of site to the surgical bed. In many instances, the deployment of this type of retractor is cumbersome because of the many arms of the retractor and the multiple movable pieces.

Reference is now made to the figures wherein like parts are referred to by like numerals throughout. Referring generally to the figures, certain embodiments disclosed herein include an expandable cannula including a distal locking mechanism. The terms “expandable cannula” and “retractor” are interchangeable within the following description.

FIG. 1 depicts an exemplary perspective view of an expandable cannula 100 with a distal locking mechanism. The device has a proximal portion 102 which is essentially tubular. Proximal portion 102 has a first diameter at first proximal end 104 and a second diameter at a first distal end 106 which are similar. Cannula 100 has an expandable distal portion 110 coupled to the proximate portion 102. The proximal and the distal portions, 102 and 110, respectively, are coupled by articulation 108 which allows the proximal portion 102 to move in relationship to the distal portion 110 to enlarge a surgeon's field of view without increasing dissection.

The distal portion has a second proximal end 112, and a second distal end 114. The distal portion 110 is expandable such that it may be in a fully contracted position, a fully expanded position, or a partially expanded position. For example, the distal portion 110, as illustrated in FIG. 1 is in a partially expanded state such that the second distal end 114 is expanded. In the contracted position the diameter of the distal portion 110 is similar to that of the proximal portion 102. In the expanded position the distal portion 110 has a diameter which is substantially larger than that of the proximal portion.

Distal Portion Embodiments:

For clarity of illustration, the distal portion embodiments below are shown without a proximal portion. However, it should be understood that a proximal portion is coupled to the distal portion as described above.

FIG. 2 depicts a perspective view of an exemplary distal locking portion 210 including a plurality of panels 220 in a partially expanded position, in one embodiment. FIG. 3 depicts the distal locking portion of FIG. 2 in a collapsed position. FIG. 4 depicts a fold out view of the distal locking portion panels 220 of FIG. 2. FIGS. 2-4 are best viewed together with the following description.

In one embodiment, the distal portion 210 is made up of a plurality of panels 220. For clarity of illustration, not all panels are labeled. For example, the panels may be “roll out panels” such that the panels of the distal portion 210 radiate outward from a proximal ring 402. Each of these panels is somewhat wider at the second distal end 214 than at an inner portion where the panel attaches to the ring 402 (i.e. where the panels are coupled together). For example, in its structural configuration, the proximal ring 402 is substantially, or is fused to, the second proximal end 212 of the distal portion 210.

In an embodiment, panels 220 are molded of a thermoplastic simultaneously with ring 402, the thermoplastic of panels 220 being relatively thick and inflexible, with thinner, flexible, “living hinge” portions 403 located at junctions between panels 220 and ring 402. “Living hinges” are known in the art of plastic molding. In this embodiment, panels 220 may also have thinned molded fold lines 405 demarcating a lateral portion 407 of each panel 220 from a central rigid portion 409 of each panel 220. Fold lines 405 permit lateral portions 407 to lie out of the plane of central rigid portion 409 such that the panels can fold to an contracted configuration having a same distal diameter 250 as proximal diameter 252 (FIG. 3), or fold to an expanded configuration having a greater distal diameter 254 than proximal diameter 252 (FIG. 1).

In an alternative embodiment, panels 220 are formed of a metal such as stainless steel, with hinges at junctions between panels 220 and ring 440.

In the contracted position, as illustrated in FIG. 3, because each of the panels 220 of the distal end portion 210 are wider distally than proximally, the distal portion of the panels overlap, allowing the distal diameter of the distal portion of the retractor to have a similar diameter to that of the proximal ring and to the proximal portion (i.e. proximal portion 102) of the expandable cannula.

In an expanded position, as illustrated in FIG. 2, the panels of the distal end of the distal portion of the retractor overlap somewhat less than in the contracted position or not at all. In this position, distal end portion 210 of the retractor has a wider diameter at the second distal end 214 than at the second proximal end 212 (e.g., proximal ring 402). This allows distal end portion 210 of the retractor to have an expanded conical configuration which when placed in the surgical bed allows for increased visualization distally with less tissue disruption proximally.

Distal Portion Locking Mechanism:

FIG. 5 is a perspective view of an embodiment 600 having a proximal portion 602, and preferably manufactured by molding from a plastic. Proximal portion 602 has two forks 604 attached to it, each of which has a hole 605 for a rivet. Proximal portion 602 is molded simultaneously with a pair of integral leaves 606, 608, and attached to the integral leaves by flexible “living hinges”, such as hinge 610, formed as thin, flexible, portion of plastic between proximal portion 602 and leaves 606, 608, configured such that the leaves may have rivets 620 (FIG. 6) to serve as a locking mechanism, in as-molded configuration. The leaves 606, 608 have slots 612 suitable for a rivet to slide in.

FIG. 6 illustrates the embodiment of FIG. 5, in expanded configuration. In this configuration, rivet 620 engages a catch-slot 622 (FIG. 5) at an expanded end of slots 612 to latch the leaves in expanded configuration.

FIG. 7 is an exterior view of an embodiment 500 having a locking ring positionable on a ladder lock element, in collapsed position. FIG. 8 illustrates a cut-away view of the embodiment of FIG. 7 along the line A-A, in collapsed position. This embodiment has a locking ring 950 formed of rigid segments 951 joined by short flexible segments 953 and pivoted 955 to panels 920.

FIG. 9 depicts a perspective cut away view of an exemplary locking mechanism 900 including a locking ring 950 that is positionable on a ladder lock element 960, in one embodiment. FIG. 10 depicts a side cut away view of the locking mechanism of FIG. 9. FIGS. 9-10 are best viewed together with the following description.

Locking mechanism 900 includes a locking ring 950 that is coupled to two separate panels 920 that are opposite one another (only one coupled panel 920 is illustrated). Locking mechanism 900 further includes two ladder lock elements 960. Each ladder lock element 960 is located at a panel that is ninety degrees from each of the panels to which locking ring 950 is coupled. Each ladder lock element 960 further includes a plurality of notches 962. Each plurality of notches 962 is adapted such that locking ring 950 may be secured within one of notches 962, as shown. Accordingly, ladder lock element 960 provides a variety of expanded positions. For example, where locking ring 950 is secured within a notch 962(1) that is toward second proximal end 912, a distal end portion 910 is not as expanded as where locking ring 950 is secured within a notch 962(2) that is toward a second distal end 914 of distal end portion 910. In an embodiment, ladder lock element 960 is molded as part of an interior surface of panel 922; in an alternative embodiment ladder lock element 960 is formed as a separate component and bonded to panel 922, in a particular embodiment the panel 922 and lock element 960 are bonded with a solvent-based glue suitable for use with a plastic from which the ladder lock element and panel 922 are formed. It should be noted that the ladder lock element effectively incorporates multiple catches, each catch adapted to secure the locking ring in a different expanded configuration.

FIG. 11 depicts a perspective cut away view of an exemplary locking mechanism 1100 including a locking ring 1150 that is positionable on a ladder lock element 1160, in one embodiment.

Locking mechanism 1100 includes a locking ring 1150 that is coupled to two separate panels 1120 that are opposite one another (only one panel 1120 is illustrated). Locking mechanism 1100 further includes two ladder lock elements 1170. Each ladder lock element 1170 is located at a panel that is ninety degrees from each of the panels to which locking ring 1150 is coupled. Ladder lock element 1170 includes a flexibly attached “zip-tie”-like structure such that locking ring 1150 may be deployed to expand panels 1120 to a plurality of diameters based upon the relation to ladder lock element 1170. The zip-tie-like structure includes multiple teeth or catches and can lock the ring at each of several positions. For example, where “zipped” toward a second proximal end 1112, locking ring 1150 has a smaller diameter, thereby placing a distal portion 1110 into a partially expanded position. However, where “zipped” toward second distal end 1114, locking ring 1150 forces panels 1120 to open to a larger diameter, thereby making the distal portion 1110 be in a more fully expanded position. Accordingly, locking element 1170 provides for a distal end portion 1110 having a variably expandable position.

In an exemplary embodiment, the distal portion of the retractor consists of four blades. Two of these blades are significantly wider at the distal end than the proximal end where they attach to the proximal ring. These wider blades serve as the cranial and caudal retraction blades of the device. The other two blades are essentially the same width from the proximal part to the distal tip or only slightly wider at the distal tip. These blades are placed in between the cranial and caudal retraction blades on either side of the retractor (medial and lateral). The purpose of these blades is to supply a “strut” which binds and stabilizes the hinge of the distal locking ring as it is deployed. Various methods of coupling the hinge of the locking ring to the strut have been described. In a preferred embodiment, a “zip-tie” method of coupling the hinge to the strut would be used. This method would allow the hinge to move distally along the strut as the distal ring unfolds, expanding the retractor into its final position.

The articulation between the proximal, “tubular” portion of the retractor and the distal “expandable” portion of the retractor allows for the proximal portion to me moved in relationship to the distal portion, allowing the surgeon to maximize distal visualization (e.g. increasing the field of view of the surgery site) while minimizing proximal dissection. The articulation may be a simple “hinge” which allows the surgeon to increase his field of view in a single plane. Alternatively, it may be a more complex articulation which allows the relationship of the proximal portion to the distal portion to change in a manner which provides the surgeon with an increased field of visualization in multiple planes. An example of a more complex articulation may be similar to that of a “universal joint” which allows an infinite number of spatial relationships between the proximal and distal portions of the retractor. This relationship may be secured in any position by means of a “friction fit”.

In one embodiment, the retractor may be made of thin metal or plastic in order to remain radiolucent and provide minimal x-ray obstruction to anatomic structures. The inner ring (i.e. the locking ring) is made of metal in order to provide sufficient hoop stresses in the expanded position to overcome resistance from the surrounding tissue and allow expansion of the distal portion of the retractor. In alternate embodiments, the inner ring (i.e. the locking ring) is made of material other than metal that has sufficient strength to overcome the resistance from the surrounding tissue and allow expansion of the distal portion of the retractor. After the surgical procedure is completed, the retractor may once again be collapsed prior to removal by disengaging the inner ring from one of its two points of contact with the inner distal diameter of the distal portion, by flexing the ring, or by cutting the ring.

FIG. 12 depicts an exemplary distal end portion 1210 including two skirt portions 1280(1), 1280(2), respectively, in an alternate embodiment. The distal end portion 1210 includes two “skirt” portions 1280(1), 1280(2). Further, each skirt portion 1280 overlaps the other, and forms one of apertures 1282(1), 1282(2) that align to the corresponding aperture 1282 of the other skirt portion 1280. Each skirt portion 1280 is attached at a pivot axis such that distal end portion 1210 may expand. Further, distal end portion 1210 includes a friction nut 1286 that allows the skirt portion to expand and remain in an expanded position.

### Twin-Leaved Embodiments With Locking Struts

An alternative embodiment 1300 (FIG. 13) has a distal end having two skirt portions 1302, 1304 each hinged to a proximal tubular portion 1306 and an internal locking strut having two rigid members 1308, 1310 (FIG. 14). In an embodiment, skirt portions 1302, 1304 are hinged to proximal tubular portion by “living hinges” 1307, 1305 formed by molding skirt portions 1302, 1304 simultaneously with tubular portion 1306 with a thin bridge of flexible plastic between tubular portion 1306 and each skirt portion. In an alternative embodiment, separate pinned hinges are used to connect skirt portions and tubular portion 1306. Each rigid member 1308, 1310 is hinged 1312, 1314 or pivoted to a skirt portion 1302, 1304. FIG. 14 is a frontal cross sectional view of the embodiment of FIG. 13, illustrating the internal locking strut with hinges or pivots. The rigid members 1308, 1310 of the locking struts are hinged together with another living hinge 1318. In an embodiment, a tab or sawtooth-shaped projection on an inside of inner skirt portion 1304 forms a catch 1310 such that the skirt portions 1302 1304 can be deployed into an expanded position by pressing rigid members and hinge 1318 until skirts are forced outwards and the rigid member crosses catch 1312. At that point catch 1312 holds the locking strut in deployed position. As seen in FIG. 15, a top cross sectional view of the embodiment of FIG. 13, illustrating an opening for surgical access between locking struts 1308-1310 and 1325.

The embodiment of FIGS. 13-15 also features a pair of rivets 1330 riding in slots 1332 that help slideably secure skirt portions 1302, 1304 to each other. Once deployed, the embodiment of FIGS. 13-15 may be collapsed back to the unexpanded state by prying catch 1312 loose from the locking struts, or by cutting the locking struts.

FIG. 16 is a cross sectional diagram of an alternative embodiment of a cannula having a distal end having two skirt portions 1602, 1604 each hinged to a proximal tubular portion 1606. In an embodiment, skirt portions 1602, 1604 are hinged to proximal tubular portion by “living hinges” 1603, 1605 formed by molding skirt portions 1602, 1604 simultaneously with tubular portion 1606 with a thin bridge of flexible plastic between tubular portion 1606 and each skirt portion. The embodiment also has an internal locking strut having two rigid members 1608, 1610 hinged together with a rivet 1612 that is also coupled to slide in a slot 1614 in a forked projection 1616 of tubular portion 1606. The internal hinged locking strut is coupled to both skirt portions 1602, 1604. When in a compressed configuration (FIG. 16), the rivet 1612 is at a proximal end of slot 1614, when in an expanded configuration (FIG. 17), rivet 1612 is at a distal end of slot 1614 with the hinge formed of rivet 1612 and rigid members 1608, 1610, overcentered such that forces acting to press the skirt portions together act to force rivet 1612 to the distal end of slot 1614, and the end of slot 1614 preventing distal movement of rivet 1612 holds the cannula in expanded configuration with the locking strut in an over-centered locked position. A detent 1615 formed in the distal end of slot 1614 helps retain the locking strut in the over-centered locked position, such that incidental jostling of the strut during surgical procedures will not collapse the cannula. The cannula may be collapsed by pulling rivet 1612 or rigid members 1608, 1610 in a proximal direction such that the hinged strut leaves the detent, passes center and collapses.

When the retractor described herein is used, a surgical wound is created by cutting skin and sealing off superficial blood vessels. A probe may be used to dissect tissue while deepening and expanding the wound to permit insertion of the retractor, during which dissection a guidewire and serial dilators may be used to expand and deepen the wound. The retractor or cannula is then inserted into the wound, during which a guidewire may be used to ensure correct placement. During insertion of the retractor, the proximal and distal portions of the retractor are kept aligned, with the distal portion in collapsed position. Once the retractor is inserted to an appropriate depth in the tissue, the distal portion is expanded to a width deemed appropriate as providing adequate access to a surgical site by the surgeon.

### Advantages of the Embodiments

The tissue retractor described in the embodiments above has several significant advantages over MIS retractors currently in use. As is common practice with many MIS retractors, this retractor may be introduced into a surgical wound over a guidewire and serial dilators with the proximal and distal portions aligned with each other. However, once appropriately placed, the retractor may then be deployed in the expanded configuration with a simple one-step mechanism which simply involves downward pressure and engagement of the inner ring. In other embodiments, the retractor is deployed by pressing the internal locking strut into a locked position. In this way, this retractor is simple to place and deploy and does not require the surgeon to manipulate many separate blades and moving parts in order to deploy and remove the retractor.

With many MIS retractors currently in use, several blades move independently of each other. While in expanded positions, there are gaps between the blades of these retractors which allow tissue to “creep” between these blades and obstruct the visual field of the surgeon. In the deployed position, this retractor assumes a substantially conical configuration in which the panels of the distal portion remain in some contact with each other providing a continuous field of view and preventing tissue “creep”.

Most current MIS retractors are inserted in a contracted position and then expand by distracting the blades away from each other and tilting the distal tips of the blades outward in order to maximize distal visualization. In this model, though there is increased visualization distally, the plane of expansion is still in a direct line which requires increased tissue dissection proximally and increased incision length in order to provide adequate visualization. The embodiments described herein have an articulation which allows the proximal and distal portions to change their relationship with each other in one or multiple planes. This articulation between the proximal and distal ends of the retractor allows for the surgeon to adjust the retractor in order to achieve maximal visualization of structures within the surgical field while minimizing proximal dissection.

Finally, because the design of current MIS retractors requires retraction of tissue with a fulcrum outside of the patient at the proximal portion of the retractor, most MIS retractors are manufactured of metal in order to overcome the significant strain involved in retracting tissue. The retractor described has the mechanical advantage of having a distal locking mechanism which obviates the need to impart significant force to adjacent tissue proximally. Thus, this retractor may be made of a much softer material such as thin metal or plastic. In this way, this retractor has the advantage of having much less material in place during the surgical procedure which allows for more direct visualization of the surgical bed and much better x-ray interpretation.

Combinations

The expandable cannula or retractor of the present invention may include a variety of features as described above, in a variety of combinations both of the cannula and its method of use. Among these combinations are:

An expandable cannula designated A including a proximal end portion having a substantially constant diameter therethrough; and a distal end portion, coupled to the proximal end portion and having a proximal end and a distal end, the distal end portion further including a plurality of panels, where the panels are coupled to the proximal end portion by hinges formed of thin flexible plastic simultaneously with molding the panels; and a mechanical locking mechanism that stabilizes the distal end portion in one of a contracted position and an expanded position.

An expandable cannula designated AA including the expandable cannula designated A, the locking mechanism including a locking ring coupled to at least two of the plurality of panel located opposite one another; and a locking element.

An expandable cannula designated AB including the expandable cannula designated A or AA wherein the locking element comprises at least one catch adapted to securing the locking ring in the expanded position.

An expandable cannula designated AC including the expandable cannula designated AB wherein the locking element includes a plurality of catches adapted such that the locking ring may be secured in a plurality of expanded positions.

An expandable cannula designated AD including the expandable cannula designated A, where the locking element includes at least one locking strut having at least two rigid members coupled together with a hinge, the rigid members each coupled to a panel of the plurality of panels and a lock for holding the rigid members.

An expandable cannula designated AE including the expandable cannula designated AD wherein the at least one locking strut is locked and stable when the hinge of the strut is configured distal to dead center.

An expandable cannula designated AF including the expandable cannula designated AD or AE wherein a rivet forming a part of the locking strut is engaged in a slot of a component coupled to the proximal end portion, and wherein when the hinge is configured distal to dead center the rivet rests against an end of the slot thereby retaining the strut in expanded configuration.

An expandable cannula designated AG including the expandable cannula designated AF wherein the slot of the component coupled to the proximal end portion further comprises a catch for retaining the strut in expanded configuration.

An expandable cannula designated AH including the expandable cannula designated A or AB, the locking element comprising a ladder lock forming a plurality of notches adapted to secure the locking ring in a plurality of positions; wherein in each position, the distal end portion is expanded to a different degree.

An expandable cannula designated AH including the expandable cannula designated A or AB, the locking element including a zip-tie element such that the locking ring may expand to a plurality of diameters based upon the relation to the zip-tie element.

A method gaining access to a surgical site designated B and including: creating a surgical wound; dissecting and dilating the wound to permit insertion of an expandable distal end portion of a cannula; the cannula having a proximal end portion having a substantially constant diameter therethrough; the distal end portion including a plurality of panels, where the panels are coupled to the proximal end portion by hinges formed of thin flexible plastic simultaneously with molding the panels; expanding the distal end portion into an expanded configuration to displace tissue and provide access through the cannula to the surgical site; and locking the distal end portion in the expanded configuration with a mechanical locking mechanism that stabilizes the distal end portion in the expanded configuration

A method designated BA including the method designated B, wherein the locking mechanism includes a locking ring coupled to at least two of the plurality of panel located opposite one another; and a locking element selected from at least one catch and a locking strut.

A method designated BB including the method designated BA wherein the locking element includes at least one catch adapted to securing the locking ring in the expanded position.

A method designated BC including the method designated BB wherein the locking element comprises a plurality of catches adapted to securing the locking ring in a plurality of expanded positions.

A method designated BD including the method designated BB wherein the locking mechanism includes at least one locking strut comprising at least two rigid members coupled together with a hinge, the rigid members each coupled to a panel of the plurality of panels and a lock for holding the rigid members.

A method designated BE including the method designated BD wherein the at least one locking strut is locked and stable when the hinge of the strut is configured distal to dead center.

A method designated BF including the method designated BD wherein a rivet forming a part of the hinge of the strut is engaged in a slot of a component coupled to the proximal end portion, and wherein when the hinge is configured distal to dead center the rivet rests against an end of the slot thereby retaining the strut in expanded configuration.

A method designated BG including the method designated BF wherein the slot of the component coupled to the proximal end portion further comprises a catch for retaining the strut in expanded configuration.

A method designated BH including the method designated B or BA, the locking element comprising a ladder lock forming a plurality of notches adapted to secure the locking ring in a plurality of positions; wherein in each position, the distal end portion is expanded to a different degree.

A method designated BI including the method designated B or BA, the locking element comprising a zip-tie element such that the locking ring may expand to a plurality of diameters based upon the relation to the zip-tie element.

### CONCLUSION

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. An expandable cannula comprising:
a proximal end portion having a substantially constant diameter therethrough; and
a distal end portion, coupled to the proximal end portion and having a proximal end and a distal end, the distal end portion comprising:
a plurality of panels, where the panels are coupled to the proximal end portion by hinges formed of thin flexible plastic simultaneously with molding the panels; and
a mechanical locking mechanism that stabilizes the distal end portion in one of a contracted position and an expanded position.

2. The expandable cannula of claim 1, the locking mechanism comprising
a locking ring coupled to at least two of the plurality of panels located opposite one another; and
a locking element.

3. The expandable cannula of claim 2 wherein the locking element comprises at least one catch adapted to securing the locking ring in the expanded position.

4. The expandable cannula of claim 3 wherein the locking element comprises a plurality of catches adapted to securing the locking ring in a plurality of expanded positions.

5. The expandable cannula of claim 1, the locking mechanism comprising at least one locking strut comprising at least two rigid members coupled together with a hinge, the rigid members each coupled to a different panel of the plurality of panels and a lock for holding the rigid members.

6. The expandable cannula of claim 5 wherein the at least one locking strut is locked when the hinge of the strut is configured distal to dead center.

7. The expandable cannula of claim 6 wherein a rivet forming a part of a hinge of the locking strut is engaged in a slot of a component coupled to the proximal end portion, and wherein when the hinge is configured distal to dead center the rivet rests against an end of the slot thereby retaining the strut in expanded configuration.

8. The expandable cannula of claim 7 wherein the slot of the component coupled to the proximal end portion further comprises a catch for retaining the strut in expanded configuration.

9. The expandable cannula of claim 2, the locking element comprising a ladder lock forming a plurality of notches adapted to secure the locking ring in a plurality of positions; wherein in each position, the distal end portion is expanded to a different diameter.

10. The expandable cannula of claim 2, the locking element comprising a zip-tie element such that the locking ring may expand to a plurality of diameters based upon the relation to the zip-tie element.

11. A method gaining access to a surgical site comprising:
creating a surgical wound;
dissecting and dilating the wound to permit insertion of an expandable distal end portion of a cannula; the cannula having a proximal end portion having a substantially constant diameter therethrough; the distal end portion comprising a plurality of panels, where the panels are coupled to the proximal end portion by hinges formed of thin flexible plastic simultaneously with molding the panels;
expanding the distal end portion into an expanded configuration to displace tissue and provide access through the cannula to the surgical site; and
locking the distal end portion in the expanded configuration with a mechanical locking mechanism that stabilizes the distal end portion in the expanded configuration.

12. The method of claim 11, wherein the locking mechanism comprises
a locking ring coupled to at least two of the plurality of panel located opposite one another; and
a locking element.

13. The method of claim 12 wherein the locking element comprises at least one catch adapted to securing the locking ring in the expanded position, and the step of locking includes securing the locking ring within the at least one catch.

14. The method of claim 13 wherein the locking element comprises a plurality of catches adapted to securing the locking ring in a plurality of expanded positions, and the step of locking includes securing the locking ring within one of the plurality of catches.

15. The method of claim 1, the locking mechanism comprising at least one locking strut comprising at least two rigid members coupled together with a hinge, the rigid members each coupled to a panel of the plurality of panels and a lock for holding the rigid members.

16. The method of claim 15 wherein the at least one locking strut is locked when the hinge of the strut is configured distal to dead center.

17. The method of claim 16 wherein a rivet forming a part of a hinge of the strut is engaged in a slot of a component coupled to the proximal end portion, and wherein when the hinge is configured distal to dead center the rivet rests against an end of the slot thereby retaining the strut in expanded configuration.

18. The method of claim 17 wherein the slot of the component coupled to the proximal end portion further comprises a catch for retaining the strut in expanded configuration.

19. The method of claim 12, the locking element comprising a ladder lock forming a plurality of notches adapted to secure the locking ring in a plurality of positions; wherein in each position, the distal end portion is expanded to a different degree.

20. The method of claim 12, the locking element comprising a zip-tie element such that the locking ring may expand to a plurality of diameters based upon the relation to the zip-tie element.
